(19) United States
(12) Patent Application Publication
(10) Pub. No.: US 2019/0350985 A1
(43) Pub. Date: Nov. 21, 2019

(54) Methods of Treating Brain Injury Using Cord Blood or a Component Thereof

(71) Applicant: Duke University, Durham, NC (US)

(72) Inventors: Joanne Kurtzberg, Durham, NC (US); Jessica Sun, Durham, NC (US); Ana Valverde Vidal, Durham, NC (US); Jesse Troy, Durham, NC (US); C. Michael Cotten, Durham, NC (US); Andrew Balber, Durham, NC (US); Daniel Laskowitz, Chapel Hill, NC (US); Arjun Saha, Durham, NC (US)

(21) Appl. No.: 16/477,110

(22) PCT Filed: Jan. 12, 2018
(86) PCT No.: PCT/US18/13623

§ 371 Date: Jul. 10, 2019

Related U.S. Application Data
(60) Provisional application No. 62/445,424, filed on Jan. 12, 2017.

(51) Int. Cl. A61K 35/51; A61K 35/28; A61K 35/44; A61P 25/28; A61K 9/00

(52) U.S. Cl. CPC A61K 35/51; A61K 35/28; A61K 9/0085; A61P 25/28; A61K 9/0019; A61K 35/44

(57) ABSTRACT

The present disclosure relates to methods of treating cerebral palsy and hypoxic-ischemic brain injury. More particularly, the present disclosure relates to methods of using cord blood or components thereof to treat cerebral palsy and hypoxic-ischemic brain injury. The present disclosure also relates to methods of assessing neuroprotective activity of a neuroprotective agent.

### CROSS REFERENCE TO RELATED APPLICATIONS

This application claims priority to U.S. Provisional Patent Application Ser. No. 62/445,424, filed Jan. 12, 2017, the contents of which are hereby incorporated by reference in their entirety.

### BACKGROUND OF THE INVENTION

### Field of the Invention

The present disclosure relates to methods of treating cerebral palsy and hypoxic-ischemic brain injury. More particularly, the present disclosure relates to methods of using cord blood or components thereof to treat cerebral palsy and hypoxic-ischemic brain injury. The present disclosure also relates to methods of assessing neuroprotective activity of a neuroprotective agent.

### Description of the Related Art

Cerebral Palsy (CP) is a condition affecting young children that causes lifelong disabilities, and typically results from in utero or perinatal injury to the developing brain, such as hypoxic insult, hemorrhage, or stroke. Affected children have varying degrees of functional impairments from mild limitations in advanced motor skills to severely limited self-mobility despite use of assistive technology, resulting in a lifelong inability to function independently. Current treatments are supportive, focusing on managing sequelae with physical therapies, medications, and surgery. However, there are no curative therapies, or therapies to address the underlying brain injury.

Hypoxic-ischemic (HI) brain injuries encompasses a wide variety of pathophysiological and molecular injuries to the brain induced by hypoxia, ischemia, or a combination of these conditions, resulting from exposure of the entire brain to dangerous reductions in oxygen (i.e., hypoxia) and/or diminished blood supply (ischemia). Neuronal death and irreversible brain injury may result from hypoxic-ischemic states if long enough in time or severe enough. Here too, the focus is on managing sequelae resulting from the HI brain injury; no therapies exist to rescue or heal the damaged brain tissue.

### SUMMARY OF THE INVENTION

In one aspect, the present invention comprises a method of treating a patient with cerebral palsy comprising administering cord blood at a dose of at least 2×10⁷ total nucleated cells/kg. In certain embodiments of this aspect of the invention, the cord blood is administered systemically. In certain aspects, the cord blood is autologous cord blood, while in other aspects it is allogenic cord blood.

In a second aspect, the present invention comprises a method of treating a patient with a hypoxic-ischemic brain injury comprising administering cord blood at a dose of at least 2×10⁷ total nucleated cells/kg. In certain embodiments of this aspect of the invention, the cord blood is administered intracerebrally, intrathecally, intranasally, intratracheally, or iraventricularly. In certain aspects, the cord blood is autologous cord blood, while in other aspects it is allogenic cord blood. In some aspects, the hypoxic-ischemic brain injury is selected from the group consisting of cerebral palsy, stroke, and hypoxic ischemic encephalopathy.

In a third aspect, present invention comprises a method of treating hypoxic-ischemic brain injury comprising administering a therapeutically effective amount of cord blood-derived CD14⁺ cells. In certain embodiments of the second aspect of the invention, the route of administration of the cord blood-derived CD14⁺ cells is intracerebral, intrathecal, intranasal, intratracheal, or intraventricular. In certain embodiments of the second aspect of the invention, the cord blood-derived CD14⁺ cells are autologous cord blood-derived CD14⁺ cells, while in other embodiments they are allogenic cord blood-derived CD14⁺ cells. In another embodiment of the second aspect of the invention, the hypoxic-ischemic brain injury is cerebral palsy.

In a further aspect, the present invention comprises a method of assessing neuroprotective activity a neuroprotective agent comprising detecting the presence of one or more secreted proteins associated with neuroprotective activity, wherein the presence of one or more secreted proteins indicates that the neuroprotective agent has neuroprotective activity. In one embodiment, the neuroprotective agent is cord blood or a component thereof. In one embodiment of this aspect of the invention, the one or more secreted proteins are selected from the group consisting of thrombospondin 1, chitinase 3-like protein 1, and metalloproteinase 9. In another embodiment of this aspect of the invention, the secreted proteins are detected by immunochemical staining with antibodies to the secreted proteins. In a further embodiment of this aspect of the invention, the cord blood component is cord blood monocytes, and in a still further embodiment of this aspect of the invention, the cord blood monocytes are CD14⁺ cells.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIG. 1. GMFM-66 scores from baseline to year 1 by randomized treatment assignment and cell dose. (A): Distribution of GMFM-66 score at baseline and 1 year in patients randomized to placebo and autologous cord blood (ACB). Lines connect the group means (circles) over time. (B): GMFM-66 change scores based on median cell doses (Precryopreservation doses: Low (LD), <3×10⁷/kg, N=16 vs. High (HD), ≥3×10⁷/kg, N=16; Infused doses: Low (LD), <1.98×10⁷/kg, N=16 vs. High (HD): ≥1.98×10⁷/kg, N=16). (C): One year Observed-Expected GMFM-66 scores in patients ≥2 years of age at baseline based on infused cell dose (Low (LD), N=10; High(HD), N=9; Placebo, N=19). (D): PDMS-2 gross motor quotient change scores based on infused cell dose (Low (LD), N=13; High (HD), N=11; Placebo, N=25). Abbreviation: GMFM-66, Gross Motor Function Measure-66.

FIG. 2. Gross motor function and brain connectivity 1 year after autologous cord blood treatment by cell dose. High dose=2×10⁷/kg, low dose <2×10⁷/kg. (A): Observed-Expected GMFM-66 scores 1 year after treatment in patients ≥2 years of age at the time of ACB infusion (low dose left; high dose right). (B): Peabody Developmental Motor Scales-2 gross motor change scores 1 year after treatment (low dose left; high dose right). (C): Change in normalized whole brain connectivity 1 year after treatment (low dose left; high dose right). Abbreviation: GMFM-66, Gross Motor Function Measure-66.

FIG. 3. Human cord blood mononuclear cells reduce death of mouse forebrain cells following OGD shock. In this and all following figures, normoxic cells were not exposed to OGD or treated with cells; values show background levels of cell death in cultures. OGD cultures were not treated with other agents and represent cell death in the absence of protective factors. A) Protection of brain cells following OGD depends on dose of CB-MNC added to slices. Slices were exposed to OGD for one hour, returned to normoxic, glucose replete conditions, and then cultured for 72 hours when cell viability was assayed by staining with DAPI and PI. PI-stained cells were counted in contiguous high power fields in the periventricular region. Graph shows mean+/−SE of PI stained as a % of DAPI-stained cells (n=3) considering the number of PI positive cell in OGD samples as 100%. Only the 25,000 cell dose group showed significant (p≤0.01) neuroprotection. B) Soluble factors from CB-CD14⁺ monocytes protect brain slice cultures after OGD shock. CB MNC were added either onto slice (dotted bar) or in medium below membrane (crosshatch bar). Statistical significances are indicated by asterisks (p<0.0001).

FIG. 4. Effect of different CB MNC subpopulations on OGD shocked brain cells in slice cultures. Experiments were performed and data is presented as in FIG. 3B. OGD shocked slices were co-cultured with CB-MNC that had been immunomagnetically depleted of the specific subpopulations or were co-cultured with immunomagnetically selected subpopulations expressing the surface antigen shown. Grid column on left shows normoxic controls. All other data from OGD shocked slices. Statistically significant differences determined by one-way ANOVA (p<0.001) compared to the OGD control are indicated by asterisks.

FIG. 5. CB CD14⁺ monocytes protect neurons following OGD shock. The average number of NeuN+ neurons, Olig2+ oligodendrocytes, and Iba1+ microglia within sequential 40× high-powered fields (HPF) located along the periventricular region was determined. Values shown are means+/−standard deviation. N=3 slices under each condition. Statistically significant differences (p<0.01) compared to the OGD control are indicated by asterisks.

FIG. 6. Effects of various supernatants and peripheral blood cell populations on OGD induced cell death in brain slice cultures. Experiments were performed as described in FIG. 3 except as noted. A) OGD shocked brain slices were treated with CB-CD14⁺ cells, with supernatants from cultured CB-CD14⁺ cells that had been exposed to culture medium conditioned by OGD shocked brain slices, or with supernatants derived from cultured CB-CD14⁺ cells as described in the examples. Dennett's multiple comparison test yielded adjusted p values for differences in cell death of 0.0001 for normoxic vs OGD, of 0.0001 for OGD vs. CD14⁺ cells added directly onto slices, of 0.0001 for OGD vs. supernatants from CD14⁺ cells exposed to OGD shocked brain supernatants, and of 0.0442 for OGD vs. supernatants from CD14⁺ cells. B) CD14⁺ or CD14 depleted PB and CB populations were added directly onto OGD shocked brain slice cultures as indicated. Statistically significant differences determined by one-way ANOVA (p<0.001) compared to the OGD control are indicated by asterisks.

FIG. 7. Protein expression analysis of CB-CD14⁺ and PB-CD14⁺ cells. Lane 1-3, represent three different samples (n=3) of CB-CD14⁺ cells and Lane 4-6 represent three different samples (n=3) of PB-CD14⁺ cells. The results confirmed enrichment of matrix metalloproteinase-9 (MMP9), thrombospondin-1 (TSP1), cystathionase (CTH), and IL-10 in CB-CD14⁺ relative to PB-CD14⁺ monocyte homogenates. GAPDH was used as loading control. Quantitative expression of each protein is shown in the table. Statistical significance (p<0.05) is shown by asterisks.

### DETAILED DESCRIPTION OF THE INVENTION

Before the disclosed processes and materials are described, it is to be understood that the aspects described herein are not limited to specific embodiments, apparati, or configurations, and as such can, of course, vary. It is also to be understood that the terminology used herein is for the purpose of describing particular aspects only and, unless specifically defined herein, is not intended to be limiting.

It is also to be understood that unless clearly indicated otherwise by the context, embodiments disclosed for one aspect or embodiment of the invention can be used in other aspects or embodiments of the invention as well, and/or in combination with embodiments disclosed in the same or other aspects of the invention. Thus, the disclosure is intended to include, and the invention includes, such combinations, even where such combinations have not been explicitly delineated.

### Definitions

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

As used herein, “treatment,” “therapy,” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient that has, or is suffering from, cerebral palsy or a hypoxic-ischemic brain injury.

As used herein, the term “disease” refers to any condition that is abnormal, such as a disorder or a structure or function, that affects part or all of a subject. In some embodiments, the disease comprises a neurological disorder. In certain embodiments, the neurological disorder comprises cerebral palsy; in other embodiments, the neurological disorder comprises a hypoxic-ischemic brain injury.

As used herein, the term “cerebral palsy” (CP) refers to any one of a number of neurological disorders that appear in infancy or early childhood and permanently affect body movement and muscle coordination but don't worsen over time. While cerebral palsy affects muscle movement, it isn't caused by problems in the muscles or nerves, but rather by abnormalities in parts of the brain that control muscle movements. The majority of children with cerebral palsy are born with it, or develop it as a result of a brain injury associated with the birthing process or in the neonatal period, although it may not be detected until months or years later. The early signs of cerebral palsy usually appear before a child reaches 3 years of age. The most common are a lack of muscle coordination when performing voluntary movements (ataxia); stiff or tight muscles and exaggerated reflexes (spasticity); walking with one foot or leg dragging; walking on the toes, a crouched gait, or a “scissored” gait; and muscle tone that is either too stiff or too floppy.

As used herein, the terms “hypoxic-ischemic brain injury” or “hypoxic-ischemic brain damage” are used interchangeably and are used to refer to any disease, disorder, or condition resulting from injuries to the brain induced by hypoxia (reduction in oxygen), ischemia (diminished blood supply), or a combination of these conditions. Hypoxic conditions may result from a number of underlying causes, including but not limited to pulmonary/respiratory dysfunction, interference by other gases (e.g. carbon monoxide), incomplete suffocation, etc. Ischemic conditions may also result from a number of underlying causes, including, but not limited to, cardiac arrest and low blood pressure, etc. These conditions lead not only to oxygen deprivation but also deprive the brain of glucose and other nutrients, and negatively impact the processes required to support brain metabolism, leading to a hypoxic-ischemic state. A hypoxic-ischemic state of sufficient severity or duration in time may lead to neuronal death and irreversible brain injury. HI brain injuries may manifest, inter alia, as seizures, spasticity, movement disorders, cognitive impairment, sensorimotor function disorders, and the like. Exemplary HI brain injuries include stroke, cerebral palsy, near drowning, cardiac arrest with prolonged resuscitation, and neonatal hypoxic-ischemic encephalopathy

### Treatment of Cerebral Palsy by Cord Blood is Dose Dependent

The inventors have surprisingly discovered that the administration of umbilical cord blood cells (CB) to children with cerebral palsy above a certain threshold dose is effective in bringing about improvement in motor function and brain connectivity in those patients. More particularly, the inventors have found the ability of cord blood to improve motor function and brain connectivity to be dose dependent. Even more particularly, positive effects are seen with a dose ≥2×10⁷ total nucleated cells/kg. Accordingly, one aspect of the invention is directed to a method of treating a patient with cerebral palsy comprising administering cord blood at a dose of at least about 2×10⁷ total nucleated cells/kg patient weight.

It is to be understood that as used herein, unless stated otherwise, the term “cord blood” is meant to encompass cord blood in any format and/or a component or mixture of components thereof, whether specifically so stated or not.

The patient may be any human or nonhuman animal. In one embodiment, the patient is human. In another embodiment, the patient is a human child under 18 years of age, or in any age range falling within this broader age range. In non-limiting examples, the patient may be a newborn, an infant 1-12 months old, 1 month to 2 years old, 1 year to 10 years old, 1 year to 8 years old, 1 year to 6 years old, 1 year to 4 years old, 1 year to 2 years old, 2 years to 10 years old, 2 years to 8 years old, 2 years to 6 years old, or 2 years to 4 years old.

The cord blood can be preserved and prepared for administration by methods known in the art. The CB may be administered to a subject by any technique known in the art, including local or systemic delivery. Routes of administration include, but are not limited to, subcutaneous, intracutaneous, intramuscular, intraperitoneal, intravenous, intrathecal, intracerebral, intraventricular, or epidural injection or implantation; topical administration; intratracheal; and intranasal administration. In some embodiments, the cord blood is administered systemically. In further embodiments, the cord blood is administered by intravenous injection.

The cord blood may be either autologous, i.e. the patient's own cord blood, or allogenic, i.e. donor cord blood.

### Treatment of Hypoxic-Ischemic Brain Injury by Cord Blood

The dosing effect observed in treating cerebral palsy suggests a suitable dose for administration of cord blood to treat brain injuries more generally, including hypoxic-ischemic brain injury. Accordingly, one aspect of the invention is directed to a method of treating a patient with a hypoxic-ischemic brain injury comprising administering cord blood at a dose of at least about 2×10⁷ total nucleated cells/kg patient weight.

Administration of the cord blood may be by any suitable route. In on embodiment of this aspect of the invention, administration is intracerebral, intrathecal, intranasal, intratracheal, or intraventricular.

The cord blood may be either autologous or allogenic. In certain embodiments, the hypoxic-ischemic brain injury is selected from the group consisting of cerebral palsy, stroke, and hypoxic ischemic encephalopathy.

### Neuroprotective Activity of Cord Blood Toward Hypoxic-Ischemic Brain Injury is Mediated by CD14⁺ Cells

CB mononuclear cells (MNC) have been tested for neuroprotective activity toward HI brain injuries. The inventors, using mouse forebrain slice cultures exposed to transient oxygen and glucose deprivation as a model of HI-induced brain damage, have discovered that CB CD14⁺ monocytes within CB MNC mediate the protection of brain cells from HI-induced damage. The inventors found a strong dose dependency in neuroprotective activity in the OGD model (over a 10-fold range of CB MNC concentration), where cells were applied directly to brain slices or in a small amount of medium directly below the slices. Moreover, the inventors discovered that while CB CD14⁺ monocytes protect brain neurons from oxygen-glucose deprivation (OGD)-induced death and suppress astrocyte activation, monocytes from adult peripheral blood (PB) and PB-CD14⁺ cells are not neuroprotective or are substantially less neuroprotective. Additionally, supernatants conditioned by CB CD14⁺ monocytes exposed to factors released from OGD-shocked brain slices were also neuroprotective, supporting a paracrine mechanism of neuroprotection by CB MNC and CB CD14⁺ cells.

Accordingly, one aspect of the invention provides a method of treating hypoxic-ischemic brain injury comprising administering a therapeutically effective amount of cord blood-derived CD14⁺ cells.

As used herein, “cord blood-derived CD14⁺ cells” means CD14⁺ cells isolated or otherwise obtained from cord blood, or the progeny of such cells, i.e. the products of expansion of such cell populations.

The cord blood, or a component thereof, such as CD14⁺ cells, can be preserved and prepared for administration by methods known in the art. With reference to cord blood, as used herein a “component thereof” refers to any part or mixture of parts of cord blood that can be isolated from the cord blood.

Administration of CD14⁺ cells confers certain advantages over the administration of CB MNC. Notably, the administration of purified CD14⁺ monocytes may afford certain safety advantages by, inter alia, limiting potential adverse reactions. Additional potential advantages include enhancement of therapeutic potency, access to therapy, use of non-HLA matched cells, and ease and accuracy in dosing.

The cord blood-derived CD14⁺ cells may be administered as a composition comprising the cells and one or more pharmaceutically acceptable carriers, adjuvants, diluents, and/or excipients.

The dose dependency in neuroprotective activity of CB MNC indicates that a more direct route of administration to brain cells may yield greater effects. Thus, while cord blood-derived CD14⁺ cells may be administered by any route of administration known in the art, in certain embodiments the route of administration is intracerebral, intrathecal, intranasal, intratracheal, or intraventricular. In some embodiments, the route of administration is intracerebral, intrathecal, or intraventricular.

In certain embodiments, the cord blood-derived CD14⁺ cells are autologous, whereas in other embodiments they are allogenic.

Cord blood-derived CD14⁺ cells may be administered to confer neuroprotective activity to a subject having any HI brain injury. In certain embodiments of this aspect of the invention, the HI brain injury is selected from the group consisting of stroke, CP, near drowning, cardiac arrest with prolonged resuscitation, and HIE. In certain embodiments, the HI brain injury is cerebral palsy. In certain embodiments, the HI brain injury is stroke. In other embodiments, the HI brain injury is HIE.

CB CD14⁺ Mediated Neuroprotective Activity is Associated with Specific Secreted Proteins

The inventors have analyzed the transcriptomes of CB CD14⁺ cells and PB CD14⁺ and discovered that they differed in the expression of many transcripts. Focusing on secreted proteins, seven transcripts were identified that could play a paracrine role in neuroprotection. Of those seven candidate genes, it was confirmed by western blotting that five of the proteins are over-expressed in CB monocytes: thrombospondin 1 (TSP-1), chitinase 3-like protein 1 (CHI3L1), matrix metalloproteinase 9 (MMP9), interleukin 10 (IL10), and inhibin, beta A (INHBA), with the first three showing the largest difference between CB and PB monocytes in western blot analysis. TSP-1 and CHI3L1 were detected in secretory granules of all CB, but not PB, monocytes, and MMP9 was abundant in a subpopulation of CB monocytes but was rare in PB monocytes. All three proteins were sequestered in cytoplasmic granules in the Golgi region, as expected for secretory proteins. Accordingly, at least TSP-1, CHI3L1, and MMP9 are associated with the neuroprotective activity conferred by CB MNC and CB CD14⁺ cells.

Thus, in one aspect, the present invention is directed to a method of assessing neuroprotective activity of a neuroprotective agent comprising detecting the presence of one or more secreted proteins associated with neuroprotective activity, wherein the presence of the one or more secreted proteins indicates that the neuroprotective agent has neuroprotective activity.

As used herein, a “neuroprotective agent” is any compound, composition, etc. that may function in a manner comparable to cord blood as disclosed herein, i.e. that is a candidate therapeutic for treating cerebral palsy and/or hypoxic-ischemic brain injury. The neuroprotective agent may be cord blood or a component thereof. The neuroprotective agent may also be cells prepared by other methods. In some non-limiting examples, the neuroprotective agent may be PB monocytes that have been treated in a manner that confers neuroprotective activity, or they may be stem cell products (e.g. IPSCs) that have been differentiated to active monocytes with neuroprotective activity such as seen with CB monocytes.

As used herein, a “secreted protein associated with neuroprotective activity” refers to a secreted protein that has been shown to be more abundant in cells conferring neuroprotective activity than in cells conferring nominal or no neuroprotective activity. In one embodiment of the invention, the secreted proteins are selected from the group consisting of thrombospondin 1, chitinase 3-like protein 1, and metalloproteinase 9.

The secreted proteins may be detected by any method know in the art, including, but not limited to, immunochemical staining with antibodies to the secreted proteins.

In one embodiment of the invention, the cord blood component is cord blood monocytes; in another embodiment, the cord blood monocytes are CD14⁺ cells.

### EXAMPLES

### Example 1: Motor Function and Brain Connectivity in Young Children with Cerebral Palsy Following Autologous Cord Blood Infusion

### Introduction

Cerebral palsy (CP) is a condition affecting young children that causes lifelong disabilities. Improved motor function has been demonstrated in animal models of ischemic brain injury and CP after administration of human umbilical cord blood cells. Evidence suggests that cord blood cells act via paracrine signaling endogenous cells to facilitate repair. After demonstrating safety, we conducted a Phase II trial of autologous cord blood (ACB) infusion in children with CP to test whether ACB could improve function.

### Materials and Methods

### Study Design

We conducted a single-center, Phase II, prospective, randomized, double-blind, placebo-controlled, crossover study of a single intravenous (IV) ACB infusion in children ages 1 to 6 years with CP at Duke University. The study was approved by the Duke Institutional Review Board and conducted under FDA IND14360.

### Participants

Eligible children were 1 to 6 years old and had CP with (a) GrossMotor Classification System (GMFCS) level 2-4 or (b) GMFCS level 1 with hemiplegia if they used their affected hand as an assist only. Children also had to have an eligible ACB unit banked at a public or private cord blood bank that was sterile, had a precryopreservation total nucleated cell count (TNCC) of ≥1×10⁷/kg, and met criteria in Table 1. Children with genetic conditions, intractable seizures, hypsarrhythmia, athetoid CP, severe microcephaly, autism without motor disability, evidence of a progressive neurologic disease or a condition that could require a future allogeneic stem cell transplant, active infection(s), impaired renal, liver or respiratory function, or a history of prior cell therapy were ineligible. Written informed consent was obtained from parent(s)/guardian(s) for patient and ACB screening and study participation.

TABLE 1
Qualifying characteristics of autologous umbilical cord blood units

| | |
|---|---|
| Characteristic | Specification |

Precryopreservation characteristics

| | |
|---|---|
| Total nucleated cell count (TNCC) | ≥1 × 10⁷/kg |
| Viability (total or CD34) | ≥80% |
| Sterility culture | Negative |
| Maternal infectious disease screening^{a} | Negative |
| Test sample available for confirmatory HLA typing | Yes |

Cord blood test sample characteristics

| | |
|---|---|
| Identity confirmation via HLA testing of subject and cord | Confirmed |
| blood sample | |
| CD34 viability | ≥60% |
| Colony forming units | Growth |

^{a}All mothers/units were tested for Hepatitis B, Hepatitis C, HIV, and syphilis. Most were also tested for HTLV I/II.
Abbreviation: HLA, Human Leukocyte Antigen.

### Randomization and Masking

Patients were randomized to the order in which they received ACB and placebo infusions, given 1 year apart. Those on the ACB arm received an infusion of ACB at baseline. Those on the placebo arm received an infusion of a placebo solution constructed to mimic the color and smell of the ACB at baseline. The placebo product consisted of TC-199+1% dimethyl sulfoxide (DMSO). Computer generated randomization was performed by The Emmes Corporation in a 1:1 ratio, stratified by age and CP typography. Only staff preparing the products were aware of the treatment assignment, and these individuals had no contact with the patients, families, providers, and examiners who were masked to the assigned treatment. Masking was achieved by covering all infusion bags with a dark bag in the laboratory and infusing a similar volume as the placebo product. Cell dose, targeted at 1-5×10⁷ cells per kilogram, was not randomly assigned, but was determined by the number of cells available in each ACB unit and the patient's weight.

### Procedures

Patients' medical records and ACB reports were reviewed. If likely to be eligible, an ACB sample was shipped to Duke for potency and viability testing. Unit identity was confirmed by low resolution Human Leukocyte Antigen (HLA)-testing of patient and ACB samples. If specifications were met, the cryopreserved ACB unit was shipped to Duke and stored under liquid nitrogen until the day of ACB infusion. Prior to enrollment, all patients were assessed by an independent examiner to confirm eligibility and assign baseline GMFCS level. On the day of ACB infusion, the product was thawed and washed in dextran 40+5% albumin (DA) and placed in 1.25 ml/kg DA for administration. Placebo infusions consisted of TC-199+1% (DMSO). The cells or placebo were administered at baseline and 1 year later in a masked manner through a peripheral IV catheter over 5-15 minutes in the outpatient setting after premedication with oral acetaminophen (10-15 mg/kg), IV diphenhydramine (0.5 mg/kg), and IV methylprednisolone (0.5 mg/kg). Subjects received maintenance IV fluids and were monitored for 2-4 hours post-infusion. Safety endpoints were incidences of infusion reactions and infections related to the study treatment. Safety assessments were conducted at 24 hours and 7-10 days after each infusion, as well as annually during return visits. Participants received traditional rehabilitation therapies per their local physicians and therapists throughout the duration of the study.

### Motor Assessments

Functional assessments were performed by trained physicians and therapists at baseline, 1-year, and 2-years. GMFCS level was assessed and motor evaluations completed, including the Peabody Developmental Motor Scales-2 (PDMS-2) and GMFM-66, a 66-item measure designed to assess gross motor function in children with CP.

### Magnetic Resonance Imaging

Magnetic resonance imaging (MRI) was performed, under moderate sedation for most participants, at baseline, 1-year, and 2-years. Diffusion weighted images were acquired on a 3 Tesla GE MR750 scanner (Waukesha, Wis.) using a 25-direction gradient diffusion encoding scheme (b=1,000 seconds/mm², 3 nondiffusion-weighted images), 70.5 ms echo time (TE), and 12,000 milliseconds repetition time (TR). Isotropic resolution of 2 mm³ was achieved using a 96×96 acquisition matrix in a field of view (FOV) of 192×192 mm². T1-weighted images were obtained with an inversion-prepared three-dimensional (3D) fast spoiled-gradient-recalled (FSPGR) pulse sequence with a 2.5 ms TE, 450 ms inversion time (TI), 6.5 ms TR, and 128 flip angle, at 1 mm³ isotropic resolution.

Whole brain connnectome analysis was based on MRI diffusion weighted images from all directions. Diffusion tensor in every voxel across the entire brain was derived, and fiber pathways tracked using fiber assignment by continuous tracking streamline tracking algorithm based on a standard fractional anisotropy (FA) threshold (0.2) to limit the pathways within the white matter. A whole-brain connectome analysis, based on all diffusion tensors, was then carried out to investigate brain connectivity and improvement among functional brain regions. These gray matter regions, termed “nodes” in the brain connectome, were defined by the JHU-DTI-MNI “Eve” atlas template, and warped into each subject's DTI image space via the Advanced Normalization Tools toolkit for a standardized processing strategy. Connectivity from any given node, or between any pair of nodes, was first measured by determining volumes of the relevant white matter fiber pathways projecting from that node or between a pair of nodes. These volumes were then further normalized by the total white matter volume (derived from a 3D FSPGR T1 weighted MRI) to remove the dependence on brain sizes due to developmental effect.

### Statistical Analysis

The primary endpoint was change in motor function from baseline to 1-year assessed by the GMFM-66. A positive change in GMFM-66 score is considered an improvement, and minimal clinically important differences (MCIDs) of medium and large effect sizes have been established. Sample size planning used estimates of this change score in untreated patients derived from a literature review (mean=6, SD=3). The study was originally planned for 60 subjects/group (n=120 total), estimated to provide 78%-97% power to detect a clinically relevant increase of 25%-35% in the mean 1-year GMFM-66 change score comparing ACB to placebo using a two-sided, equal-variance t test and 5% Type I error rate.

Two unplanned interim analyses were conducted for the primary endpoint. Efficacy stopping rules were designed to preserve the overall Type I error rate at 5% using an alpha spending function, ƒ(t)=min(α*t³, α). The null hypothesis of no difference between treatment groups was not rejected at either analysis. A simulation study for conditional power conducted after the first interim analysis suggested potential benefit in continuing the trial. However, due to slow accrual, the trial was closed when enrollment reached n=63. When all subjects had completed the 1-year assessment and after verifying assumptions, the test of the primary hypothesis was performed using an equal-variance, two sample t test with a critical value of 2.00, which maintained an overall two-sided cumulative alpha of 0.05 across interim looks with the final sample size. Results of the interim analyses were reviewed by the primary investigators. Study personnel conducting outcome assessments were not informed of the results.

All analyses (performed using SAS versions 9.3 and 9.4) followed the intention-to-treat principle. The primary endpoint was compared between ACB and placebo using an equal-variance t test. Additional analyses, involving comparison of outcomes by dose between ACB and placebo 1 year after baseline and among all patients 1 year after treatment with ACB, used the t test, Wilcoxon rank sum test, Fisher's exact test, or Spearman correlation as appropriate. We defined high- and low-dose categories using the cohort median dose. For analyses from baseline to 1 year, the median dose was calculated for the 32 patients randomized to the treatment arm (3.0×10⁷/kg precryporeservation, 1.98×10⁷/kg infused). For analyses of the composite cohort (all children 1 year post ACB infusion), we used the median infused dose for all 63 patients, 2×10⁷/kg.

### Results

### Characteristics of Patients and ACB Units

63 patients were enrolled and randomized to receive an initial infusion of ACB (n=32) or placebo (n=31) with a crossover to the alternate infusion 1 year later. Subjects' etiology of CP was classified as: periventricular leukomalacia (n=17), in utero stroke/bleed (n=27), ischemic injury (n=7), other multifactorial causes (n=12). Of these 12 patients, etiologies and MRI findings were highly variable and included two patients who were born premature, one with a porencephalic cyst, three with white matter abnormalities, five with normal MRIs, and one of unclear etiology. One-third of patients had moderately severe GMFCS levels (3-4) at study entry. Treatment groups were balanced with respect to age, sex, race, type, and severity of CP (Table 2).

ACB units were retrieved from 16 international cord blood banks. All subjects received all infusions as intended. The median precryopreservation TNCC of banked ACB units was 4.9×10⁸. To achieve the target TNCC dose of 1-5×10⁷/kg, the entire ACB unit was used in 31 patients. In the other 32 patients for whom the cell dose from the whole ACB unit would have exceeded the dosing range, a portion of the ACB unit was used for infusion and the remainder was cryopreserved and stored for potential future use. Post-thaw, a median of 2×10⁷ TNCC/kg were administered (range 0.38-5.03×10⁷/kg), containing a CD34⁺ dose of 0.5×10⁵/kg (range 0.05-4.9×10⁵/kg). ACB unit characteristics are shown in Table 2.

TABLE 2
Characteristics of patients and autologous cord blood units by randomized treatment assignment and cell dose

| Randomized assignment | | | |
|---|---|---|---|
| Autologous | | Infused cell dose | |
| Cord blood | Placebo | Low | High |
| group | group | (<2 × 10⁷/kg) | (≥2 × 10⁷/kg) |
| (N = 32) | (N = 31) | (N = 31) | (N = 32) |

Patient characteristics

| | | | | | | | | |
|---|---|---|---|---|---|---|---|---|
| Age, years - median (range) | 2.1 | (1.1-6.2) | 2.3 | (1.1-7.0) | 2.5 | (1.1-7.0) | 2.1 | (1.1-5.3) |
| Sex - no. (%) | | | | | | | | |
| Male | 20 | (62.5) | 22 | (71) | 20 | (64.5) | 22 | (68.8) |
| Female | 12 | (37.5) | 9 | (29) | 11 | (35.5) | 10 | (31.3) |
| Race - no. (%) | | | | | | | | |
| White | 27 | (84.4) | 28 | (90.3) | 26 | (83.9) | 29 | (90.6) |
| Non-white | 5 | (15.6) | 3 | (9.7) | 5 | (16.1) | 3 | (9.4) |
| Type of cerebral palsy - no. (%) | | | | | | | | |
| Hypotonic quadraplegia | 1 | (3-1) | 3 | (9.7) | 2 | (6.5) | 2 | (6.3) |
| Spastic diplegia | 6 | (18.8) | 6 | (19.4) | 7 | (22.6) | 5 | (15.6) |
| Spastic hemiplegia | 15 | (46.9) | 15 | (48.4) | 12 | (38.7) | 18 | (56.3) |
| Spastic quadraplegia | 10 | (31.3) | 7 | (22.6) | 10 | (32.3) | 7 | (21.9) |
| GMFCS level^{a} - no. (%) | | | | | | | | |
| I/II | 21 | (65.6) | 21 | (67.7) | 18 | (58.1) | 24 | (75.0) |
| III/IV | 11 | (34.4) | 10 | (32.3) | 13 | (41.9) | 8 | (25.0) |
| Baseline GMFM-66 score - mean (SD) | 48.9 | (16.2) | 52.0 | (15.7) | 48.9 | (20.3) | 51.9 | (10.1) |

Cord blood characteristics - median (range)^{b}

| | | | | | | | | |
|---|---|---|---|---|---|---|---|---|
| Collection volume, ml | 66 | (4.5-146) | 64 | (5.7-150) | 56 | (4.5-146) | 83 | (20-150) |
| Pre-cryo TNCC, ×10⁸ | 4.4 | (1.1-15.5) | 5.1 | (1.9-12.6) | 2.8 | (1.1-10.3) | 7.1 | (2.9-15.5) |

| | | | | | | | |
|---|---|---|---|---|---|---|---|
| Cell dose infused, ×10⁷/kg | 2.0 | (0.8-4.8) | — | 1.5 | (0.4-1.9) | 3.1 | (2.0-5.0) |
| CD341 dose infused, ×10⁵/kg | 0.60 | (0.11-3.90) | — | 0.40 | (0.05-2.00) | 0.80 | (0.20-3.90) |
| CFU dose infused, ×10⁵/kg | 3.91 | (0.04-36.21) | — | 4.0 | (0-36.2) | 4.6 | (0-20.0) |

^{a}GMFCS = Gross Motor Function Classification System. Difference between dosing groups is not statistically significant.
^{b}All cord blood characteristics except CFU dose are statistically significant between doing groups (p < .01) and not statistically different between randomized groups.
Abbreviation: TNCC, total nucleated cell count.

### Safety of ACB Infusions

Infusions of thawed ACB and placebo products, both containing DMSO, were well tolerated, and there were no serious adverse events related to the infusions. One patient had transient infusion reactions consisting of hives +/−low-grade fever after both placebo and ACB infusions, successfully treated with additional diphenhydramine. Despite negative precryopreservation cultures, one ACB unit grew b-hemolytic streptococcus from a sample of the thawed unit. That patient was not treated with antibiotics and did well.

### GMFM-66 Results

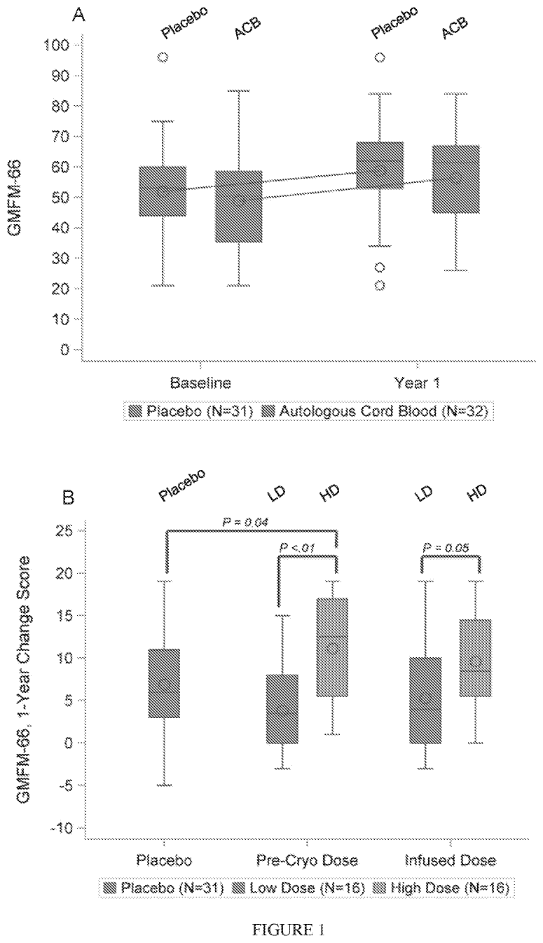

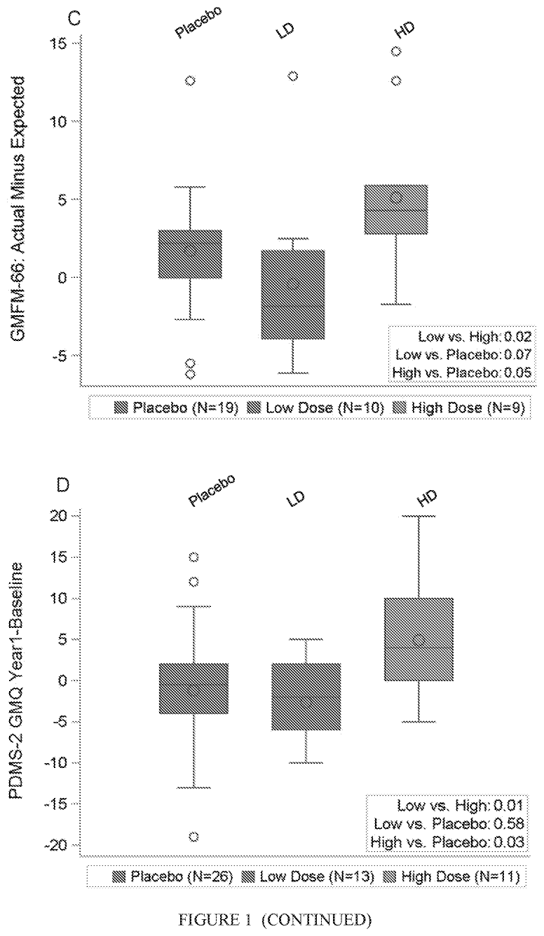

Change in GMFM-66 score from baseline to 1-year was the primary endpoint. The observed mean change in GMFM-66 score was 7.5 points (SD 6.8) in the ACB group and 6.9 points (SD 5.5) in the placebo group (t_{df}=61, =0.36, p=0.72, FIG. 1). Of note, both groups improved more than expected based on patients' age and GMFCS level at study entry. However, subjects randomized to ACB who were treated with TNCC doses above the median precryopreservation or infused doses of 3×10⁷/kg and 1.98×10⁷/kg, respectively, demonstrated statistically significant, clinically meaningful improvement in GMFM-66 change scores, above MCIDs, compared with subjects who received lower cell doses (p<0.01 for precryopreservation dose, p=0.05 for infused dose) or placebo (p=0.02 for precryopreservation dose) (FIG. 1B). Cell doses were not associated with baseline age or type/severity of CP (Table 2). In the placebo group, change from baseline to 1 year was not associated with the precryopreservation cell dose available in the subjects' ACB unit. CD34 cell doses were not associated with motor improvement.

To examine the effect of cell dose and to adjust response for the natural history of expected gains based on baseline GMFCS levels and GMFM-66 scores of each subject, we used published percentiles (Hanna S E, et al., Phys Ther 88:596-607 (2008)) to compare the actual GMFM-66 score change to the predicted change. The difference between the observed 1-year GMFM-66 score and the predicted 1-year GMFM-66 score was then calculated. Since percentile values are only available for children ≥2 years, this analysis included the 38 patients who were ≥2 years old at study entry. There was no significant difference in the median observed-expected difference in GMFM-66 scores at 1 year in patients randomized to ACB (n=19; 1.7; IQR −2.5 to 4.5) versus placebo (n=19; 2.2; IQR 0.0 to 3.0; p=0.99). However, in an exploratory analysis, subjects who received a TNCC ≥2×10⁷/kg (n=9) improved a median of 4.3 points (IQR 2.8-5.9) greater than expected, and this change was statistically significantly different from that observed in subjects who received <2×10⁷/kg (n=10; median −1.9, IQR −3.9 to 1.7; p=0.02) or placebo (n=19; median 2.2, IQR 0.0 to 3.0; p=0.05, FIG. 1C), with improvement above the MCID of large effect size.

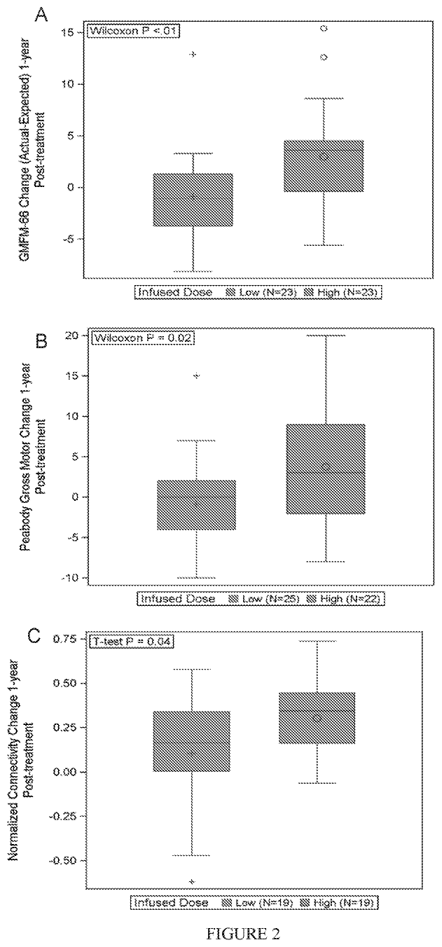

We then used the 2-year data to further explore the effect of cell dose by comparing the difference between observed and expected GMFM-66 scores 1 year after ACB infusion in all subjects who were ≥2 years old when they were treated (n=46), regardless of when the infusion was given (baseline or 1 year). In this analysis, the dose relationship from the primary analysis was confirmed: subjects who received ≥2×10⁷ cells per kg (n=23) improved a median 3.6 points (IQR −0.4 to 4.5) greater than expected, whereas subjects who received <2×10⁷/kg did not improve beyond expectation (median −1.1, IQR −3.7 to 1.3, p=0.003, FIG. 2A).

### PDMS-2 Results

At 1 year post initial treatment (ACB vs. placebo), 50 patients were eligible for analysis of the PDMS-2 Gross Motor Quotient, which assesses gross motor skills in young children from birth to 72 months of age. Of note, eight subjects excluded from analysis of observed-expected GMFM-66 scores due to age (<2 years) were included in the PDMS analysis. The median change from baseline did not differ significantly between randomized groups (ACB 1.0, IQR −4.5 to 4.5 vs. placebo −0.5, IQR −4.0 to 2.0; p=0.39); but analysis of the treated group by infused cell dose confirmed the observation from the GMFM-66 analysis, with greater improvement in the high dose group (FIG. 1D). The dose finding was consistent in the 2-year analysis, when all subjects treated with ACB were analyzed by infused TNCC (>/<2×10⁷/kg); subjects receiving high doses showed statistically significant improvement compared with subjects receiving lower doses (median [IQR]: high-dose 3.0 [−2.0 to 9.0] vs. low-dose 0 [−4.0 to 2.0], p=0.02, FIG. 2B).

### Imaging Results

We explored relationships between motor response, total brain connectivity, and cell dose. Accurate anatomical image parcellation could not be obtained in approximately one-third of subjects due to injury that distorted normal brain morphology, leaving 23 treated and 15 placebo patients with evaluable connectivity data (n=38). There were no statistically significant differences in CP type, GMFCS level, or age between patients with and without analyzable images.

There was a moderate correlation between change in GMFM-66 score and total connectivity 1 year after baseline in all analyzable subjects (n=38, Spearman r=0.53; 95% CI: 0.25, 0.73; p<0.001). In this cohort, total connectivity change was not related to baseline GMFCS level, typography of CP, or sex, but was inversely correlated with age (Spearman r=−0.52; 95% CI: −0.72, −0.23; p=0.001). In the 2-year analysis when all evaluable subjects were examined by cell dose, patients who received ≥2×10⁷ TNCC/kg (n=19) demonstrated a statistically significant greater increase in normalized whole brain connectivity 1 year after treatment than children who received lower doses (n=19; p=0.04, FIG. 2C). In the sensorimotor network, nodes with significant increases in connectivity that correlated with improvement in GMFM-66 scores included the pre- and post-central gyri, basal ganglia, and brain stem.

### Discussion

We observed that children who received higher cell doses (≥2×10⁷/kg infused) demonstrated superior gains in both whole brain connectivity and motor function 1 year after infusion of ACB. The median change in GMFM-66 score in the high-dose group (8.5, IQR 5.5-14.5) exceeded that of the low-dose (4, IQR 0.0-10.0) and placebo (6, IQR 3.0-11.0) groups by more than established MCIDs, indicating a statistically significant and clinically meaningful difference between dosing groups. These responses were not correlated with age or type, etiology, or severity of CP.

Important relationships were also detected via whole brain connectome analysis of MRI/DTI data, an objective measure of whole brain connectivity including the motor network, suggesting that improvements in motor function result from increased or new connectivity induced by paracrine signaling of ACB cells. We confirmed that increased total brain connectivity is correlated with increased motor improvement. Furthermore, we also showed that compared with children who received a low cell dose, children who received a dose ≥2×10⁷/kg demonstrated a greater increase in both normalized total brain connectivity and changes in the sensorimotor network, including the pre- and post-central gyri, deep gray matter, and brain stem (FIG. 2C) 1 year post-treatment with ACB.

The improvements in motor function and brain connectivity demonstrate the neuroprotective activity of CB dosed a level of at least 2×10⁷ total nucleated cells/kg. In view of this neuroprotective activity, treatment with cord blood at such a level can be expected to be effective on a broader category of brain injury, including hypoxic-ischemic brain injuries.

As the hypothesized mechanism of action—that cells contained in ACB act on endogenous cells in the brain via paracrine signaling to enhance brain connectivity and thus function—does not require engraftment or integration of infused cells, donor cord blood cells may be equally efficacious.

### Example 2: Cord Blood Monocytes Rescue Brain Neurons from Hypoxic-Ischemic Injury and Express Unique Secretory Proteins

### Introduction

Mononuclear cell (MNC) products prepared from human umbilical cord blood (CB) are candidate therapeutics for treatment of brain injuries in which hypoxic-ischemic (HI) injury is a major pathogenic component. Patients with cerebral palsy, neonatal hypoxic-ischemic encephalopathy (HIE), and acute ischemic stroke have been treated with intravenously administered CB-MNC products in early safety and feasibility trials. Preclinical studies suggest that CB-MNCs promote favorable resolution of brain injury following HI injury by releasing paracrine neurotrophic and anti-inflammatory factors that stimulate repair by host cells. Studies using various animal and culture systems have implicated different CB-MNC subpopulations as contributing to neuroprotection.

We used oxygen and glucose deprived (OGD) organotypic mouse forebrain slice cultures to assess which cell types in CB-MNC enhance brain tissue repair and the mechanisms by which they do so. In this well-established model of HI brain injury, excised brain slices were allowed to stabilize in culture, were exposed to hypoxic shock and glucose deprivation, and then were returned to normoxic conditions with glucose containing media. This model can be used to test cell products for neuroprotective activity following OGD-induced neuronal death.

### Materials and Methods

### Animals

All experiments were performed in accordance with Duke University Institutional Animal Care and Use Committee's policies and followed approved protocols. C57BL/6 mice (The Jackson Laboratory) were maintained in Duke facilities under direct veterinary supervision. Animals had ad libitum access to food and water in a temperature-controlled room under a 12-hour light: 12-hour dark illumination cycle.

### Oxygen-Glucose Deprivation (OGD) of Brain Slice Cultures

Organotypic forebrain slice cultures were prepared as described in the literature. Briefly, brain slices were cultured under controlled atmosphere conditions on top of cell impermeable membranes in contact with culture medium. Preliminary studies showed that cellularity changed in these cultures over three weeks, but the number of neurons in the periventricular zone was stable 10-12 days after cultures were initiated, and OGD experiments were performed during this time-period. Slices were exposed to medium without glucose in an oxygen-free gas mixture for one hour, returned to normoxic, glucose replete conditions, and incubated for 72 hours before further analysis.

Treating OGD-Shocked Cultures with Cells or Supernatants

To test the protective activity of human CB or PB cell populations, 2.5×10⁴ cells were added directly onto each brain slice immediately after OGD shock. Alternatively, cell populations were added indirectly to the tissue culture medium below the membrane supporting slices. In some experiments, conditioned medium from a cell population was added below the membrane instead of cells. Cell death and/or the cellular composition of periventricular region of the slice cultures were compared to control slices not treated with cells 72 hours after OGD treatment.

### Assessment of Cell Death Following OGD

Slices were transferred to medium containing propidium iodide (PI, 2.0 μg/mL, Sigma) and incubated for 30 minutes to stain necrotic cells, washed thoroughly with PBS, fixed with 4% paraformaldehyde (PFA) containing 4,6-diamidino-2-phenylindole (DAPI). Slides were coded, and percentage of total cells (DAPI staining) that were necrotic (PI staining) in multiple sequential images of the periventricular region was determined. Each slide was analyzed by an investigator blinded to the identity of the experimental material using a Leica SP8 upright confocal microscopy (Leica Microsystems, IL, USA) and ImageJ and Plugin Cell Counter (NIH Image, USA) software.

### Immunohistological Analysis of Cell Populations in Slice Cultures

Brain slices were fixed in 4% PFA and blocked in phosphate buffered saline (PBS) containing 3% heat-inactivated horse serum, 2% bovine serum albumin (BSA), and 0.25% triton-X-100 overnight. Primary antibody was prepared in 2% BSA, 0.25% triton X-100 in PBS. Slides were incubated in antibody for 24-48 hours and subsequently washed once for 30 minutes and twice for 1 hour in PBS. Secondary antibody was prepared in 2% BSA in PBS. Slides were incubated 24 hours, subsequently washed once 30 minutes and twice for 1 hour and mounted with Vectashield (Vector Labs, CA, USA). Images were analyzed as described for PI staining.

### Isolation of Human Umbilical Cord and Adult Peripheral Blood Cell Populations

Freshly collected human umbilical cord blood was provided by the Carolinas Cord Blood Bank at Duke, an FDA licensed public cord blood bank that accepts donations of cord blood collected after birth from the placentas of healthy term newborns after written informed consent from the baby's mother. Also with maternal informed consent, cord blood units not qualifying for banking for transplantation were designated for research and made available for this study. Peripheral blood (PB) was obtained via venipuncture from healthy adult volunteer donors. Human samples were obtained using protocols approved by the Duke University Institutional Review Board. Mononuclear cells were isolated from CB and PB by density centrifugation using standard Ficoll-Hypaque technique (GE Healthcare) then treated with 0.15M NH₄Cl to lyse residual erythrocytes and washed in phosphate-buffered saline (PBS).

### Immunomagnetic Cell Isolation for OGD Experiments

Specific sub-populations were isolated or removed from CB-MNC or PB-MNC by immunomagnetic sorting using EasySep cell kits for human CD34⁺, CD3⁺, CD14⁺ and CD19⁺ cells (Stemcell Technologies, Vancouver, Canada, Catalog #18096, #18051, #18058 and #18054 respectively) following the manufacturer's directions. Flow-through fractions from positive selection columns were re-run through the columns to increase the purity of targeted populations. A sample of each cell preparation was analyzed by flow cytometry to determine cellular composition.

### Carboxyfluorescein Succinimidyl Ester (CFSE) Labeling

CD14⁺ CB monocytes were stained with 5 μM CFSE, V12883, green fluorescence (Life Technologies) as described by the manufacturer to track cells in tissue slices.

### RNA Isolation and Microarray Analysis

RNA isolation and microarray analysis were carried out as described previously using 54,675 probe set Affymetrix GeneChip Human Transcriptome Array 2.0 microarrays and Partek Genomics Suite 6.6 (Partek Inc., St. Louis, Mo.) software for analysis (Saha, A et al., JCI Insight 1(13):e86667 (2016)). Table 3 outlines the number of donors and the characteristics of the donors used for each chip. The experimental methods used to purify CD14⁺ monocytes from donor samples for subsequent microarray analysis and to prepare cells used for RNA extraction were as follows: For Method 1, MNC fractions were prepared by centrifugation on Ficoll, treated with NH₄Cl to remove erythrocytes, and CD14⁺ cells were immunomagnetically selected using Easysep [Stem Cell Technologies, Vancouver BC]; for Method 2, CB was processed by Method 1 and then flow sorted at the Duke University Comprehensive Cancer Center Flow Cytometry Facility using PeCy7-mouse anti-human CD14 monoclonal antibody (Becton Dickenson catalog 562698) to obtain a purified CD14⁺ preparation. Cells were maintained at 0-4° C. during all procedures including flow sorting; for Method 3, after NH₄Cl lysis, MNC preparations were incubated on ice with PeCy7-mouse anti-human CD14 (BD catalog 562698), FITC-mouse anti-human CD3 (BD catalog 555339), and FITC-mouse anti-human CD235a (BD catalog 559943) antibodies. Cell suspensions were then flow sorted twice, for each sample an initial enrichment sort was followed by a purity sort to yield a CD14⁺CD235a⁻CD3⁻ population. Cells were maintained at 0° C.−4° C. during all procedures, including flow sorting.

TABLE 3
Donor Characterization

| Experiment | Source | Gender | Ethnicity | Hours^{a} | Method^{#} | Purity^{b} |
|---|---|---|---|---|---|---|
| 1213 | PB-1 | Male | Caucasian | Fresh | 1 | 92 |
| 1213 | PB-2 | Male | Asian Indian | Fresh | 1 | 85 |
| 1213 | PB-4 | Male | Asian Indian | Fresh | 1 | 95 |
| 1213 | CB-A | Male | Caucasian | 18 | 1 | 87 |
| 1213 | CB-B | Male | Asian Indian | 15 | 1 | 92 |
| 1213 | CB-C | Male | Asian Indian | 9 | 1 | 91 |
| 314 | CB | Male | Caucasian | 13 | 2 | 95 |
| 714 | CB-1 | Male | Caucasian | 21 | 3 | 99.4 |
| 714 | CB-2 | Female | African American | 16 | 3 | 97.5 |
| 714 | CB-3 | Male | Caucasian | 13 | 3 | 97.4 |
| 714 | CB-4 | Male | Caucasian | 10 | 3 | 99 |
| 714 | PB-2 | Female | Asian Indian | Fresh | 3 | 99 |
| 714 | PB-4 | Male | Caucasian | Fresh | 3 | 97.9 |
| 714 | PB-5 | Male | Caucasian | Fresh | 3 | 99 |
| 714 | PB-6 | Male | Asian Indian | Fresh | 3 | 99.2 |

^{a}Hours after collection of CB sample when isolation of CB-CD14⁺ cells was initiates.
^{b}Percent of total population expressing CD14 determined by flow cytometry as previously described.

Quantitative Polymerase Chain Reaction (qPCR) of Candidate Gene Expression

We prepared CB and PB-MNC preparations by centrifuging blood in Sepmate tubes (Stem Cell Technologies, Vancouver, BC) on a ficoll gradient as described by the manufacturer. This removed most erythroid cells without using NH₄Cl. CD14⁺ cells. Monocytes were purified from these MNC preparations without any further manipulation using CD14 Microbeads (Miltenyi Biotech, San Diego, Calif.) as described by the manufacturer. Expression of candidate genes identified from microarray analysis was measured by quantitative polymerase chain reactions using the methods and primers described by Scotland et al. (Scotland, P et al., Cytotherapy 19(6): 771-82 (2017)).

### Western Blotting

Western blotting was carried out as previously described (Saha, A et al., JCI Insight 1(13):e86667 (2016)) using commercially obtained antibodies.

### Statistical Analysis

All comparisons were performed by one-way analysis of variance (ANOVA) followed by post hoc analysis with Bonferroni correction. Mean differences were considered significant if p<0.05 was computed.

### Results

CB-MNC Protect Forebrain Slice Culture Cells from OGD Induced Death

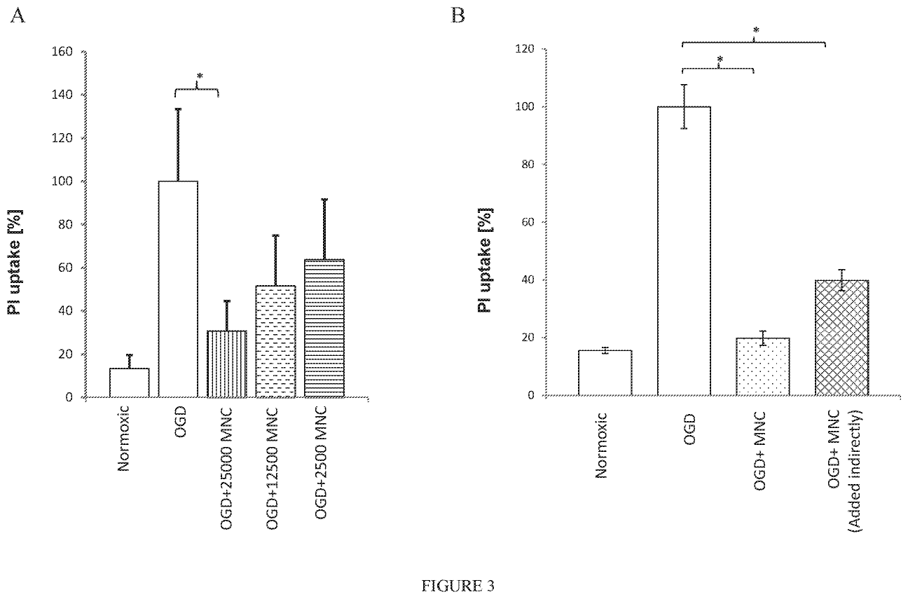

A significant percentage of cells in forebrain slice cultures became permeable to PI during the three days following OGD shock. In each of three experiments using CB-MNC from different CB donors and at least two brain slices per experimental condition adding 25,000 CB-MNC to the surface of an OGD-shocked brain slice reduced the percentage of PI-stained necrotic brain cells significantly (p<0.01) to 10-20% of the of percentage in OGD-shocked control slices cultured without CB cells. These percentage of dead cells in OGD-shocked cultures protected by CB-MNC approached background cell death in normoxic cultures. FIG. 3A shows that the protective effect of CB-MNC was dose dependent between 2,500 to 25,000 CB-MNC per slice experiment with only 25,000 CB-MNC/slice yielding statistically significant (p<0.01) protection. Accordingly, we used this dose of cells for all other experiments.

Soluble Factors Released by CB-MNC Participate in Protection of Brain Cells after OGD

To determine whether the protective effects of CB-MNC are dependent on direct cell-cell contacts between CB-MNC and OGD shocked brain slices, we added CB-MNC to the medium below the membrane instead of directly onto the OGD-shocked slices. This prevented direct contact between CB-MNC and brain cells, but permitted agents released or secreted by CB-MNC to interact with forebrain cells through the 0.4 μm pores. We used more CB-MNC cells in these experiments than when we added cells directly to slices to compensate for dilution of factors by the culture medium. In the experiment shown in FIG. 3B, adding 1.25×10⁵ CB-MNC below the membrane significantly (p<0.05) reduced brain cell death to 39.8±7.6% [mean+/−SD; n=3 wells of 2-3 slices per condition] of the value seen in OGD shocked slices maintained without CB-MNC. Similar results were obtained in two separate experiments performed using this protocol. Thus, a portion of the effect of neuroprotection by CB-MNC after OGD was mediated through paracrine signaling.

We applied 2.5×10⁴ CFSE-labeled CD14⁺ CB monocytes to OGD shocked brain slices and examined the slices by fluorescence to follow the fate of the human cells. The cells that we found on the tissue slice after 72 hours had grown in size, had put out numerous projections, and appeared to be highly activated.

### CD14⁺ Monocytes are Primarily Responsible for the Protective Effects of CB-MNC.

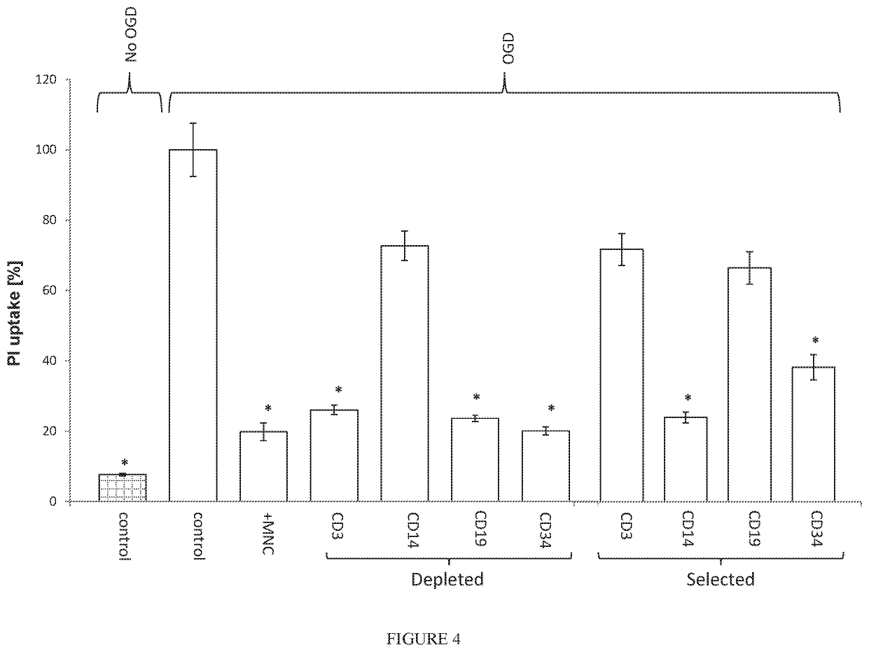

To identify what types of cells mediate the protective effects of CB-MNC following OGD, we depleted CB-MNCs of specific cell types using immunomagnetic techniques from 3 cord blood donors. Depleted cell sub-populations were added to forebrain slice cultures immediately after OGD shock, and cell death was measured after 72 hours. Results from a representative experiment are shown in FIG. 4. Depletion of CD14⁺ monocytes reduced the protective activity of CB-MNC 3.5-fold. Depletion of CD3⁺, CD19⁺ or CD34⁺ cells did not significantly reduce the protective activity of CB-MNC.

We also used positive selection to isolate various subpopulations of CB-MNC and tested these directly for protective effects in slice cultures. We applied 2.5×10⁴ cells directly to each slice immediately after OGD shock, and assayed cell death as before. Neither selected CD3⁺ nor CD19⁺ cells protected brain cells under these conditions (FIG. 4). Selected CD14⁺ cells protected brain cells from death following OGD as efficiently as CB-MNC cells at this cell concentration (FIG. 4). Selected CD34⁺ cells also showed some protection (38.9±3.6% [mean+/−SD; n=3], p<0.05 compared to controls); this protection was significantly less (p<0.05) than that shown by CD14⁺ monocytes.

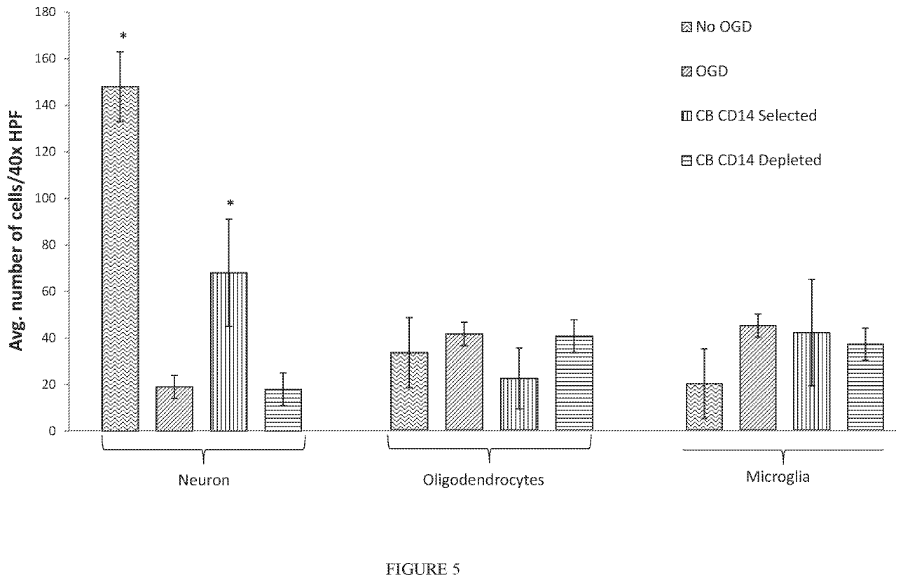

Immunohistochemical analysis demonstrated that CB-CD14⁺ monocytes preserved neurons and dampened astrocyte activation following OGD-shock. The increase in cell necrosis detected with PI following OGD shock was mirrored by a large decrease in the NeuN-stained neuronal nuclei per field in shocked cultures (FIG. 5); neurons appeared largely apoptotic. GFAP-stained astrocytes in OGD-shocked slices became hypertrophic and extended multiple processes taking on the characteristic activated morphology. Since GFAP is a cytoplasmic protein, this morphological change made it difficult to quantitate changes in the number of astrocytes, as it was difficult to resolve individual cells. Iba-1 staining is also cytoplasmic, but individual microglia did not show as dramatic changes on morphology. The percentage of oligodendrocyte nuclei stained by anti-Olig2 or of microglia did not change significantly following OGD (FIG. 5). Selected CB-CD14⁺ preserved NeuN staining neurons and dampened astrocyte activation (FIG. 5). CB-MNC preparations depleted of CD14⁺ monocytes did not protect neurons or dampen astrocyte activation (FIG. 5).

CB-CD14⁺ Monocytes Release Soluble Neuroprotective Factors when Exposed to Supernatants from OGD Shocked Brain Slices

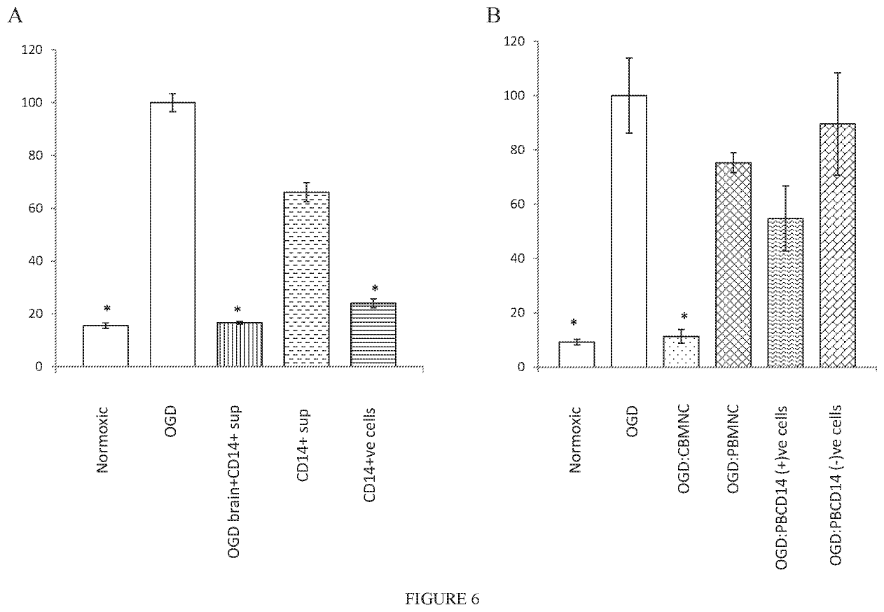

We used the trans-well slice culture system to examine whether CB-CD14⁺ monocytes release neuroprotective factors constitutively or whether exposure to damaged brain tissue stimulates release of such factors (FIG. 6A). Brain slice cultures were exposed to OGD, and supernatants were collected from below the membrane. CB-CD14⁺ monocytes were then exposed to these OGC-shock supernatants, and the supernatants from these cultured monocytes were collected. These cell free supernatants were then added back to brain slice cultures immediately after OGD shock to determine if supernatants protected brain cells over the following 3 days. Results were compared to supernatants derived either from CB-CD14⁺ monocytes cultured for 3 days; supernatants from CB-CD14⁺ monocytes exposed to medium conditioned by OGD-shocked brain cultures. Supernatants from CB-CD14⁺ monocytes exposed to medium conditioned by OGD-shocked brain cultures were more protective than those from monocytes not exposed to shocked brain products (FIG. 6A).

CD14⁺ Monocytes from PB Give Minimal Protection from OGD Shock

Because PB-MNC are plentiful in patients with unresolved HI induced injuries, we explored differences in neuroprotective activity between the two monocyte populations as an initial step to identifying specific neuroprotective mechanisms in CB-CD14⁺ monocytes. Applying PB-MNCs, CD14⁺ depleted PB-MNC, or selected CD14⁺-PB monocytes directly to forebrain slice cultures did not forestall brain cell death (FIG. 6B), loss of neuron content, or astrocyte activation following OGD shock as effectively as CB-CD14⁺ monocytes.

Whole Transcriptome Analysis of CD14⁺ Monocytes Derived from CB and PB

Based on the difference in neuroprotective activity of CB and PB-CD14⁺ monocytes in the OGD model, we evaluated potential gene products involved in neuroprotection as expressed in CB-CD14⁺ vs. PB monocytes. We compared the transcriptomes of CB and PB-CD14⁺ monocytes using whole transcriptome microarray chips to try to identify these gene products. Table 3 provides demographic information on the donors. The preparation of CD14⁺ cells used for the analysis was as described above.

We analyzed seven adult PB donors and seven CB donors in two experiments, four donor pairs on one chip (Experiment 714) and three on a second (Experiment 1213). Another CB-CD14⁺ monocyte donor was analyzed on a third chip (Experiment 314), but not compared to PB donors; this dataset was used to confirm mRNA expression levels of CB samples.

CB and PB-CD14⁺ monocytes have unique mRNA expression profiles. An MS5 analysis for the two chips comparing CB and PB monocyte gene expression was conducted. This analysis scores the normalized fluorescence signal for each probe set as expressed or not expressed compared to background. In both experiments 714 and 1213 in which CB and PB cells were compared, about 18,500 probe sets detected transcripts expressed in all CB samples, about 20,000 probe sets detected transcripts expressed by all PB samples, and about 24,000 probe sets did not detect expressed transcripts in either cell population. Thus, there was reasonable agreement in these gross expression parameters between the two analyses. As expected there was more variability in the number of probes detecting mixed expression (expression in only 1 or 2 of 3 CB or PB donor samples in experiment 1213 and 1, 2, or 3 of 4 donor samples in experiment 714). The critical observation was that in both experiments CB and PB monocytes differentially expressed many transcripts. The largest differences in expression were in transcripts expressed by all CB and no PB samples and in transcripts expressed by some PB and no CB samples; these gene products were consistently expressed only in CB or PB monocytes respectively. Transcripts expressed by all CB and no PB samples or some PB samples, transcripts expressed by some CB and no PB samples or some PB samples, and more remotely transcripts expressed by some CB and all PB represented potential candidate genes contributing to the enhanced neuroprotective activity of CB monocytes.

RMA provided quantitative information about the magnitude of differential gene expression for each transcript. A heat map presentation of the data analysis for Experiment 1213, for example, showed that CB and PB-CD14⁺ monocytes differentially expressed 1553 transcripts. Of these, 475 probes detected transcripts expressed at higher levels in CB-CD14⁺ monocytes, and another 1078 probes detected transcript more highly expressed in PB-CD14⁺ monocytes. CB and PB-CD14⁺ monocytes fall into discrete populations defined by these differentially expressed transcripts.

We mined the RMA expression data to identify over expressed CB monocyte transcripts that encode secreted proteins. Such proteins could account for the ability of CB-CD14⁺ monocytes to protect brain neurons through the transwell membrane. We defined candidates as genes that (1) encoded secreted proteins or proteins that directly synthesized secreted products, (2) were over expressed in CB monocytes relative to PB monocytes in both microarray experiments, (3) were also highly expressed by CB monocytes in Experiment 314, and (4) were differentially expressed in confirmatory quantitative PCR analysis using RNA from additional donors. This screen minimized the likelihood of variations in expression arising from differences in donor characteristics or methods used to purify CD14⁺ monocytes for RNA extraction. Seven candidates emerged from this analysis (Table 4).

TABLE 4^{a}
Seven Candidate Genes Encoding Secreted Factors Over-Expressed by CB Compared to PB-CD14⁺ Monocytes.

| | | | Chip Experiment 1213 (n = 4) | | | Chip Experiment 714 (n = 3) | | |
|---|---|---|---|---|---|---|---|---|
| | | | | Fold- | | | Fold- | |
| Gene Symbol | Gene Title | Probe set | p-value | difference | Note | p-value | difference | Note |
| CTH | cystathionase | 217127_at | 5.70E−04 | 41.9 | CB only | 1.18E−08 | 105.1 | CB > PB |
| | | 206085_s_at | 3.44E−03 | 14.3 | CB only | 8.14E−08 | 26.0 | CB only |
| CHI3L1 | chitinase 3-like 1 | 209395_at | 1.33E−02 | 12.6 | CB only | 4.99E−02 | 5.0 | CB only |
| | | 209396_s_at | 1.83E−02 | 10.6 | CB only | 1.75E−01 | 2.8 | CB = PB |
| THBS1 | thrombospondin 1 | 215775_at | 6.59E−03 | 3.1 | CB > PB | 4.71E−03 | 2.6 | CB > PB |
| | | 201107_s_at | 1.74E−02 | 3.5 | CB > PB | 5.84E−04 | 3.7 | CB only |
| | | 201109_s_at | 1.19E−04 | 32.1 | CB > PB | 5.56E−02 | 9.3 | CB > PB |
| | | 201108_s_at | 7.37E−04 | 20.5 | CB only | 8.98E−03 | 8.4 | CB > PB |
| | | 201110_s_at | 3.78E−05 | 22.2 | CB > PB | 1.98E−01 | 4.3 | CB > PB |
| | | 235086_at | 2.88E−04 | 35.0 | CB > PB | 1.78E−02 | 8.4 | CB > PB |
| | | 239336_at | 2.24E−03 | 8.8 | CB only | 4.42E−03 | 5.5 | CB only |
| MMP9 | matrix | 203936_s_at | 1.18E−03 | 13.8 | CB > PB | 1.17E−02 | 5.4 | CB > PB |
| | metallopeptidase 9 | | | | | | | |
| IL10 | interleukin 10 | 207433_at | 1.51E−02 | 2.5 | CB > PB | 3.44E−03 | 2.8 | CB > PB |
| VEGF-A | vascular endothelial | 210512_s_at | 1.18E−03 | 3.6 | CB > PB | 1.91E−01 | 2.0 | CB = PB |
| | growth factor -A | 212171_x_at | 2.77E−04 | 4.4 | CB > PB | 3.38E−02 | 2.2 | CB > PB |
| | | 210513_s_at | 2.09E−03 | 4.1 | CB > PB | 4.97E−02 | 1.9 | CB = PB |
| | | 211527_x_at | 1.35E−03 | 5.1 | CB > PB | 5.31E−02 | 2.9 | CB > PB |
| INHBA | inhibin, beta A | 227140_at | 6.07E−03 | 10.5 | CB only | 4.36E−02 | 14.3 | CB > PB |
| | | 210511_s_at | 2.28E−02 | 2.3 | CB > PB | 6.75E−02 | 3.9 | CB > PB |

| | | |
|---|---|---|
| 204926_at | not detected | not detected |

^{a}All probes sets detecting each candidate genes in both microarrays are shown. Cord and peripheral blood donors are described in Table 3. p values are derived from RMAD analysis. Notes show MS5 analysis and indicate whether transcripts were detected exclusively in CB [CB only] or in both CB and PB-CD14⁺ cells [CB > PB].

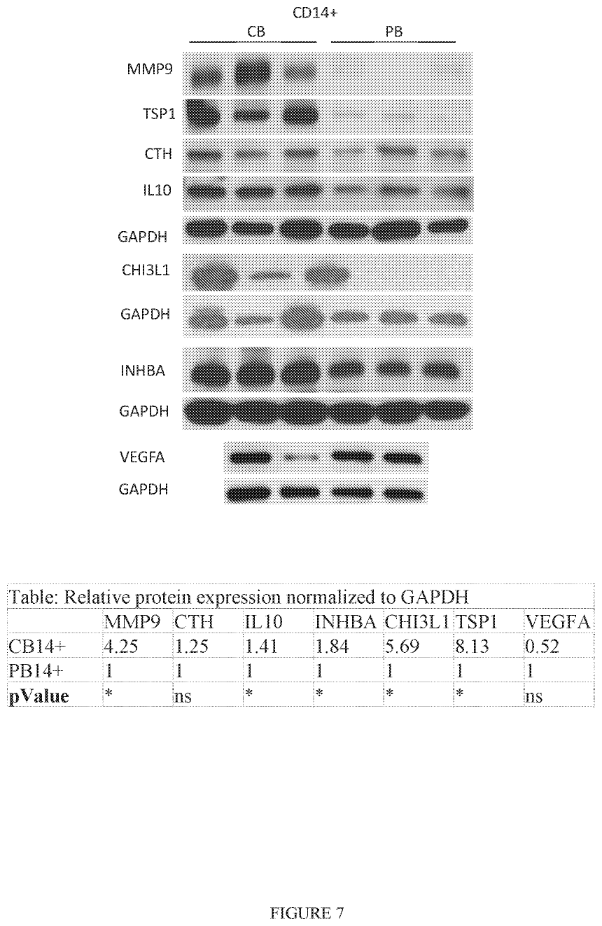

We next analyzed whole cell extracts of CB and PB-CD14⁺ monocytes by quantitative western blotting to determine the intracellular content of the proteins encoded by these candidate genes (FIG. 7). Densitometry showed that the amount of CTH in PB and CB monocytes was not significantly different; VEGFA was more abundant in PB-CD14⁺ monocyte homogenates, although not statistically significant. CB monocyte homogenates contained significantly (p≤0.03) more IL10, INHBA, MMP9, CHI3L1, and TSP1 than PB-CD14⁺ monocytes.

We also used immunocytochemistry to determine how MMP9, CHI3L1, and TSP1 proteins were expressed within CB and PB-CD14⁺ monocyte populations. CHI3L1 and TSP1 were more strongly expressed in CB than PB-CD14⁺ monocytes and these two proteins were present in virtually all CD14⁺ monocytes. CB monocytes also expressed more MMP9 than PB monocytes, but in this case, expression was confined to a subpopulation of CD14⁺ monocytes that was less common in PB monocyte populations. All three proteins were sequestered in cytoplasmic granules in the Golgi region, as expected for secretory proteins.

### Discussion

We have demonstrated that CB-MNC, specifically the CD14⁺ cells, protect neurons from death after OGD shock. CB-CD14⁺ monocytes also dampen the astrogliosis that results from OGD shock. Our data indicate that CB CD14⁺ monocytes used as a therapeutic agent may have similar effects whether administered alone or as a component of CB-MNC.

Comparative gene expression analysis revealed that CB monocytes overexpress a discrete group of transcripts compared to PB monocytes. We analyzed secretory molecules over expressed in CB monocytes based on literature implicating paracrine factors in CB-MNC mediated repair of brain tissue and on the transwell experiments discussed above showing that much of the neuroprotection in the OGD model resides in soluble factors. Proteins encoded by five (CHI3L1, TSP1, MMP9, IL10, and INHBA) of the seven candidate genes we initially identified were more abundant in homogenates of CB than PB monocytes. TSP1, CHI311, MMP9, IL10, and INHBA can all promote tissue repair in models of tissue, including brain, repair. The other two proteins, VEGFA and CTH, were expressed by CB monocytes, but steady state levels of intracellular protein detected by western blots were not higher in CB than PB monocytes. CHI311, TSP1, and MMP9 showed the largest difference in western blots analysis, and immunochemical staining confirmed that CB monocytes have more of these three proteins in cytoplasmic granules, presumably secretory granules, than PB monocytes. Thus, CHI311, TSP1, and MMP9 may be particularly important in paracrine mechanisms by which CB monocytes protect brain neurons from OGD.

The ability to discriminate monocytes that have neuroprotective function from monocytes that do not, based on immunochemical staining with antibodies to CHI3L1, TSP1, and MMP9 or other methods, provides a potency assay for CB products used in the treatment of HI injury. Most CB monocytes, but few or no PB monocytes strongly express TSP1 and CHI3L1 in cytoplasmic, secretory granules. In addition, a subpopulation of monocytes that expresses MMP9 is more abundant in CB than PB. Fully understanding the potency of a CB unit in treating HI brain injury will require describing how functionally distinct monocyte subpopulations arise in CB and PB; TSP-1, CHI311, and MMP9 are useful markers in clarifying these issues.

We found a strong dose dependency over a ten-fold range of CB monocyte concentration in the OGD model even though cells were directly applied to slices. In hematopoietic stem cell transplant patients receiving intravenously injected CB-MNC grafts, most CB cells are removed in the lungs and other organs during first pass circulation. Womble et al. (Mol Cell Neurosci., 59:76-84 (2014)) found that the beneficial activity of intravenously injected CB-MNC in a rat stroke model resided in the CD14⁺ monocyte population, however how many CB-MNC or monocytes actually reach the brain following intravenous injection in patients with HI-induced brain injury is unknown. Our results using the OGD shocked brain slice model may be particularly pertinent to protocols in which cell products are delivered directly to the brain, by, e.g., intracerebral, intraventricular, or intrathecal administration. Administering purified CD14⁺ monocytes as a therapeutic agent locally in the brain may afford a safety advantage compared to freshly thawed, heterogeneous CB-MNC products.

1-23. (canceled)

24. A method of treating a patient with cerebral palsy comprising administering cord blood at a dose of at least about 2×10⁷ total nucleated cells/kg.

25. The method of claim 24, wherein the cord blood is administered systemically.

26. The method of claim 24, wherein the cord blood is autologous cord blood.

27. The method of claim 24, wherein the cord blood is allogenic cord blood.

28. A method of treating a patient with a hypoxic-ischemic brain injury comprising administering cord blood at a dose of at least about 2×10⁷ total nucleated cells/kg or administering a therapeutically effective amount of cord blood-derived CD14⁺ cells.

29. The method of claim 28, wherein the cord blood or the cord blood-derived CD14⁺ cells are administered intracerebrally, intrathecally, intranasally, intratracheally, or iraventricularly.

30. The method of claim 28, wherein the route of administration is intracerebral, intrathecal, or intraventricular.

31. The method of claim 28, wherein the cord blood or cord blood-derived CD14⁺ cells are autologous cord blood or cord blood-derived CD14⁺ cells.

32. The method of claim 28, wherein the cord blood or cord blood-derived CD14⁺ cells are allogenic cord blood or cord blood-derived CD14⁺ cells.

33. The method of claim 28, wherein the hypoxic-ischemic brain injury is cerebral palsy.

34. The method of claim 28, wherein the hypoxic-ischemic brain injury is stroke.

35. The method of claim 28, wherein the hypoxic-ischemic brain injury is hypoxic ischemic encephalopathy.

36. The method of claim 28, wherein the administration is of cord blood at a dose of at least about 2×10⁷ total nucleated cells/kg.

37. The method of claim 28, wherein the administration is of a therapeutically effective amount of cord blood-derived CD14⁺ cells.

38. A method of assessing neuroprotective activity of a neuroprotective agent comprising detecting the presence of one or more secreted proteins associated with neuroprotective activity, wherein the presence of the one or more secreted proteins indicates that the neuroprotective agent has neuroprotective activity.

39. The method of claim 38, wherein the neuroprotective agent is cord blood or a component thereof.

40. The method of claim 38, wherein the one or more secreted proteins are selected from the group consisting of thrombospondin 1, chitinase 3-like protein 1, and metalloproteinase 9.

41. The method of claim 38, wherein the one or more secreted proteins are detected by immunochemical staining with antibodies to the secreted proteins.

42. The method of claim 39, wherein the cord blood component is cord blood monocytes.

43. The method of claim 42, wherein the cord blood monocytes are CD14⁺ cells.
